(19) United States
(12) Patent Application Publication
(10) Pub. No.: US 2005/0238597 A1
(43) Pub. Date: Oct. 27, 2005

(54) Topical treatment of acne, seborrheic dermatitis, and oily skin with formulations containing histamine antagonists

(76) Inventors: John P. McCook, Frisco, TX (US); Thomas J. Stephens, Plano, TX (US)

Correspondence Address: THEODORE C. JAY;16 NORTH CHATSWORTH AVE., APARTMENT 600, LARCHMONT, NY, 10538, US

(21) Appl. No.: 10/832,174

(22) Filed: Apr. 26, 2004

(51) Int. Cl. A61K 7/135

(52) U.S. Cl. 424/66

(57) ABSTRACT

Topical compositions containing histamine antagonists together with pharmaceutically acceptable salts and dermatological acceptable carrier vehicles are applied to the skin of human patients to inhibit sebaceous gland activity associated with acne, seborrheic dermatitis (seborrhea), and cosmetic oily skin conditions.

### TECHNICAL FIELD

This invention relates to topical compositions containing histamine antagonists and specifically H-1 antagonists, commonly referred to as antihistamines or antihistaminics, for inhibiting sebaceous gland activity associated with acne, seborrheic dermatitis (seborrhea), and cosmetic oily skin conditions.

### BACKGROUND OF THE INVENTION

Acne, seborrhea or seborrheic dermatitis are general classes of skin diseases which are characterized by an abnormal function of the sebaceous glands in human skin. Additionally, non-pathogenic seborrhea, or, what is referred to as excessively oily skin or cosmetic oily skin is a condition that affects a significant number of the population and is marked by unusually high daily sebum flow, particularly on the face.

### DESCRIPTION OF THE INVENTION

In the search for a safe and effective sebossuppressive agent that could be used topically, applicants screened a number of classes of pharmaceuticals that are commonly in use for various pharmacological activities, including topical antihistamines. Antihistamine drugs or H-1 antagonists are not known to have any effect on the sebaceous gland or the production of sebum.

Applicants have discovered that certain topical antihistamine preparations typically sold over the counter and used to relieve itching, in particular, diphenhydramine hydrochloride [1-2%] and tripelennamine hydrochloride [0.5-2.0%], when used in various formulations, can be used to reduce generation of sebum, and moreover, once sebum is reduced, subsequent treatment of acne, seborrhea or dermatitis by known conventional methods is more effective and less objectionable to patients so treated.

Screening studies of candidate sebosuppressive agents, including topical antihistamines, began with human female subjects between the ages of 18 and 50 with a self declared history of oily facial skin, confirmed by clinical assessment and by the use of the Sebumeter SM 815®, manufactured by Courage & Khazaka Electronics GmbH. The Sebumeter measurement is based on grease-spot photometry. A special tape becomes transparent in contact with the sebum on the skin surface. For the determination of the sebum, the measuring head of the Sebumeter cassette is inserted into the aperture of the device, where the transparency of the tape is measured by a light source sending light through the tape which is reflected by a little mirror behind the tape. A photocell measures the transparency. The light transmission represents the sebum content on the surface of the measuring area. A microprocessor calculates the result, which is shown on the display in μg sebum/cm² of the skin.

Typically, qualified subjects would have a multi-year history of problem oily skin. These subjects were asked the question, “How long after the first washing of your face in the morning does it take for your face to look and feel oily?” Subjects that were cross-validated for oily skin by the Sebumeter and clinical grading answered four hours or less to this question.

A series of pilot clinical studies evaluated the topical use of diphenhydramine HCl at 2% in a hydoalcoholic gel formulation shown in Exhibit 1 below

### EXHIBIT 1

### Oil Control Lotion

### Formula #24-53A

| Ingredient | % w/w |
|---|---|
| Natrosol HHR 250 | 1.00 |
| (hydroxyethylcellulose) | |
| Camphor | 0.40 |
| Ethanol SD 40, 190 proof | 50.00 |
| Diphenhydramine HCl (Sigma) | 2.00 |
| Phenonip (Clariant Corp.) | 1.00 |
| Deionized Water | 45.60 |
| Total | 100.00 |

A control formulation, identical to the formula 24-53A listed above, but without diphenhydramine, was evaluated with the active formula among ten subjects with self-assessed oily facial skin for a period of ten days. A randomized assignment of active treatment or control formulation was made in this double-blind placebo controlled pilot study. Subjects were screened for eligibility based upon history of oily skin, agreement to use no other topical products during the study other than the test products and a mild facial cleansing bar provided, and answers to a questionnaire of various inclusion and exclusion criteria. Subjects applied the test products once a day in the morning to the forehead and cheeks after washing the face with a mild facial cleansing bar.

Results of this pilot study indicated moderate sebum suppressive activity for the antihistamine gel. Self-assessment ratings at the end of this ten day study showed a 44.2% average reduction for the active gel (p<0.06) and 19% (p<0.03) for the control. However, despite the large average reduction in facial oil for the formulation with diphenhydramine HCl, variability was high for the active gel with one subject claiming no reduction, one subject claiming slight reduction and three subjects claiming a drastic reduction. Sebumeter readings were taken at baseline and at the final visit of the subjects on day 10. Sebumeter readings were recorded for 5 facial locations for each subject, three on the forehead and one on each cheek. The analysis of the sebumeter readings showed a slight average reduction of roughly 10% for either the active or control formulation. Despite the large range in subject responses, the subject self-assessment ratings were seen as more indicative of true sebosupressive activity versus the Sebumeter readings for this small, short duration study.

A second pilot study evaluated the sebosuppressive activity of diphenhydramine HCl, 2% in a silicone gel lotion containing penetration enhancing glycols, ethanol, a silicone emulsion base (System 3; Collaborative Laboratories), and surface-treated starch as shown below in Exhibit 2:

### EXHIBIT 2

### Oil Control Lotion

### Formula #24-109

| Ingredient | % w/w |
|---|---|
| Natrosol HHR 250, (1% Solution) | 45.00 |
| System 3AM-200 (Collaborative) | 20.00 |
| Methyl Sulfonyl Methane (MSM) | 5.00 |
| Dry Flo Starch (National Starch | 10.00 |
| & Chemical) | |
| Hydrolite-5 (Dragoco) | 5.00 |
| Ethanol SD 40, 190 proof | 10.00 |
| Diphenhydramine HCl (Sigma) | 2.00 |
| Phenonip | 0.30 |
| Citrus Sea fragrance 40080T | 0.07 |
| Deionized Water | 2.63 |
| Total | 100.00 |

Formulation 24-109 shown above was evaluated by 9 subjects in a ten day pilot study with the study protocol similar to the first pilot study but a second formulation without diphenhydramine HCl was not included. Sebosuppessive activity was evaluated based on subject self assessment at baseline and after ten days of once/day treatment. Results of this pilot study showed an average sebosuppression of 39.3% with a high level of significance (p<0.007).

A third pilot study was conducted with ten subjects that evaluated once daily application of a test formulation in the evening after washing the face with a mild cleansing bar. The study protocol was otherwise similar to the second pilot study. Both Sebumeter and subject self-assessment measures were used at baseline and at the end of the study (day 7).

The treatment prototype again contained 2% diphenhydramine HCl in a silicone gel lotion as shown below in Exhibit 3

### EXHIBIT 3

### Oil Control Lotion

### Formula #24-173

| Ingredient | % w/w |
|---|---|
| Natrosol HHR 250, (1% Solution) | 45.00 |
| System 3 AM-200 (Collaborative) | 20.00 |
| Ethoxydiglycol (Trivalin SF) | 4.00 |
| Dry Flo Starch (National Starch | 10.00 |
| & Chemical) | |
| 1,3-Butylene Glycol | 4.00 |
| Glycerin | 2.00 |
| Ethanol SD 40, 190 proof | 10.00 |
| Diphenhydramine HCl (Sigma) | 2.00 |
| Phenonip | 0.60 |
| Citrus Sea fragrance 40080T | 0.07 |
| Deionized Water | 2.33 |
| Total | 100.00 |

Results of this third pilot study showed an average reduction in facial oil between baseline and the 7 day readings of 23% (p<0.003) based on subject self-assessment and 15.9% (p<0.02) based on Sebumeter readings.

Three separate pilot clinical studies demonstrated moderate sebosuppression of facial sebum among female subjects who applied an oil control formulation containing diphenhydramine HCl, 2%, once daily for a period of 7-10 days.

Diphenhydramine HCl is a first generation H-1 antagonist that has been used for over 50 years to treat cold and allergy symptoms, motion sickness and mild insomnia. H- 1 antagonists block histamine release from receptors present on skin and smooth muscle tissues (e.g., capillaries within the nasal and bronchial mucosa).

The mechanism of action for the sebosupressive activity of diphenhydramine is unknown. However, histamine release is presumptively involved in the production of excessively oily skin. The total level of histamine in facial skin is relatively high when compared with other dermal areas and significantly higher than the histamine content of other bodily tissues or fluids with perhaps the exception of the lungs. For example, the histamine level of facial skin is 5 times the level of abdominal skin It is also well known that histamine release increases the secretions from gastric, salivary, sweat, lacrimal and bronchial glands, as well as the pancreas.

The effect of histamine on sebaceous gland activity has not been studied. High levels of fatty acids produced by the enzymatic breakdown of triacylglycerols and possibly other byproducts may initiate an irritant reaction within the sebaceous glands with subsequent histamine release and a further increased production of sebum as a defensive mechanism against the fatty acid/histamine response. The cycle of fatty acid irritant->histamine release->increased sebum production->fatty acid irritant may be responsible for maintaining excessively oily skin throughout much of the teenage and adult life of men and women who complain of this problem. As stated above, diphenhydramine HCL (Benadryl®) is a first generation H-1 antagonist. We would expect other first generation H-1 antagonists as well as second and third generation H-1 antagonists or combination H-1 and H-2 antagonists to have sebosuppressive activity.

Examples of first, second and third generation antihistamines are as follows:

### First Generation H-1 Antagonists

### Diphenhydramine (Benadryl®)

Chlorpheniramine
Dimenhydrinate (Dramamine®)
Brompheniramine
Promethazine
Hydroxazine (Atarax®)
Triprolidine
Trimeprazine
Cyproheptadine (Periactin®)
Phenyltoloxamine
Phenindamine
Pyrilamine

The above antihistamines are listed as the base compound but may typically be used as a salt (e.g., diphenhydramine hydrochloride, chlorpheniramine maleate, pyrilamine tannate, hydroxazine pamoate, etc.). First generation or so-called classic antihistamines generally fall into one of six chemical classes: Alkylamines, Ethanolamines, Ethylenediamines, Piperazines, Phenothiazines, or Piperadines.

### Second Generation H-1 Antagonists (“Non-Sedating Antihistamines”)

Terfenadine (Seldane®)
Fexofenadine(Allegra®)
Loratadine(Claritin®)
Cetirizine (Zyrtec®)
Clemastine (Tavist®)
Disloratidine (Clarinex®)
Ranitidine

The second generation antihistamines are listed as base compounds but some may be used as a salt (e.g. Cetirizine HCl)

### Third Generation H-1 And Combination Antagonists

tecastemizole
levocetirizine
Azelastine (Astelin(®)
Astimazole
H-1/NK-1 dual antagonists
Doxepin HCL (Zonalon®)

The above H-1 antagonists are listed for illustration purposes and are not inclusive of all antihistamine compounds which are typically heterocyclic or nitrogen compounds having at least one benzene ring and further characterized by inhibiting the contraction of smooth muscles induced by the administration of histamine or inhibiting the release of histamine by stimulated mast cells.

Other histamine antagonists that preferentially block histamine release from gastric parietal cells, known as H-2 inhibitors (e.g.,Pepcid®, Tagamet®, Zantac(®) or block histamine release from neurons and are known as H-3 inhibitors (e.g., desferriozamine, denbufylline, donepezil) are specific for gastric or neuron receptor cells, respectively. However, H-1, H-2, and H-3 receptors are present in the skin and recent studies have shown that both H-1 and H-2 antagonists improve the skin's barrier repair properties and prevent epidermal hyperplasia induced by barrier disruption. H-3 antagonists had no effect on barrier repair.

In order to confirm that H-1 antagonists, as a class have sebosuppresssive activity rather than a specific sebosuppressive activity only for diphenhydramine HCl, a fourth pilot clinical was conducted with a topical formulation containing tripelennamine hydrochloride, a first generation antihistamine classified as a non-specific histamine antagonist with low to no anticholinergic activity. Tripelennamine has a long history of topical and systemic use and is currently approved for OTC topical use in the U.S. at concentrations of 0.5-2%.

The fourth pilot clinical study evaluated the twice daily (morning and evening) topical application of a lotion formulation containing tripelennamine HCl, 2% versus a control lotion with no antihistamine as shown in Exhibits 4 and 5 below. A randomized assignment of active treatment or control formulation was made in this double-blind placebo controlled pilot study. Subjects were screened for eligibility based upon history of oily skin associated with mild-moderate acne, agreement to use no other topical products during the study other than the test products and a mild facial cleansing bar provided, as well as answers to a questionnaire of various inclusion and exclusion criteria. A total of 15 subjects (8-test product; 7-control product) treated their facial skin twice daily for 29 days. The subjects assessed the degree of facial oiliness daily on an eight point analog scale utilizing a self-assessment criterion described above for the other pilot studies. Each subject recorded the degree of oily skin on a daily log and the daily score was compared to the original baseline score to calculate the % reduction in oil. For example, if subject 1, on day 4 had a self-assessment score of 2.0 and a baseline score of 1.0, the % reduction is calculated as the treatment score minus the baseline score divided by the baseline score. In this example, the % reduction=4.0-2.0/2.0×100=100% reduction.

### EXHIBIT 4

### Oil Control Lotion

### Formula #25-147

| Ingredient | % w/w |
|---|---|
| Natrosol HHR 250, (1% Solution) | 45.00 |
| System 3 AM-200 (Collaborative) | 20.00 |
| Ethoxydiglycol (Trivalin SF) | 4.00 |
| 1,3-Butylene Glycol | 4.00 |
| Glycerin | 2.00 |
| Ethanol SD 40, 190 proof | 10.00 |
| Tripelennamine HCl (Sigma) | 2.00 |
| Phenonip | 0.60 |
| Citrus Sea fragrance 40080T | 0.07 |
| Deionized Water | 12.33 |
| Total | 100.00 |

### EXHIBIT 5

### Oil Control Lotion

### Formula #25-155

| Ingredient | % w/w |
|---|---|
| Natrosol HHR 250, (1% Solution) | 45.00 |
| System 3 AM-200 (Collaborative) | 20.00 |
| Ethoxydiglycol (Trivalin SF) | 4.00 |
| 1,3-Butylene Glycol | 4.00 |
| Glycerin | 2.00 |
| Ethanol SD 40, 190 proof | 10.00 |
| Phenonip | 0.60 |
| Citrus Sea fragrance 40080T | 0.07 |
| Deionized Water | 14.33 |
| Total | 100.00 |

Results of this fourth pilot clinical showed a statistically significant reduction in facial oil for the test product containing tripelennamine HCl beginning on day 4 (p=0.0303) and every day from day 4 through the end of the study on day 29 (p=0.0008) with mean daily oil reduction ranging from 58.3% to 140.4% compared to the baseline. Subjects in the control group rated their treatment as having a positive affect on oil production as well but the average daily reduction was much lower for the control ranging from zero to 125% compared to baseline and the sebosuppression was inconsistent throughout the study with nearly half the days showing no statistically significant reductions. A comparison of the mean sebum reductions by day for 29 days for the topical antihistamine formula (Formula #25-147; Treatment A) versus the control formulation (Formula ∩25-155; Treatment B) is shown in Exhibits 6 & 7 below:

### EXHIBIT 6

Average percent change from baseline
TRT = A

| VISIT | N obs | N | Mean |
|---|---|---|---|
| 1 | 8 | 8 | 58.3333333 |
| 2 | 8 | 8 | 108.3333333 |
| 3 | 8 | 8 | 99.5833333 |
| 4 | 8 | 8 | 93.7500000 |
| 5 | 8 | 8 | 93.7500000 |
| 6 | 8 | 8 | 104.5833333 |
| 7 | 8 | 8 | 87.9166667 |
| 8 | 8 | 8 | 92.0833333 |
| 9 | 8 | 8 | 127.9166667 |
| 10 | 8 | 8 | 134.1666667 |
| 11 | 8 | 8 | 121.6666667 |
| 12 | 8 | 8 | 100.8333333 |
| 13 | 8 | 8 | 120.0000000 |
| 14 | 8 | 8 | 124.1666667 |
| 15 | 8 | 8 | 161.6666667 |
| 16 | 8 | 8 | 121.6666667 |
| 17 | 8 | 8 | 125.8333333 |
| 18 | 8 | 8 | 109.1666667 |
| 19 | 8 | 8 | 123.3333333 |
| 20 | 8 | 8 | 119.1666667 |
| 21 | 8 | 7 | 112.3809524 |
| 22 | 8 | 8 | 100.8333333 |
| 23 | 8 | 7 | 115.2380952 |
| 24 | 8 | 8 | 121.6666667 |
| 25 | 8 | 8 | 127.9166667 |
| 26 | 8 | 8 | 115.4166667 |
| 27 | 8 | 8 | 115.4166667 |
| 28 | 8 | 8 | 140.4166667 |
| 29 | 8 | 8 | 140.4166667 |

### EXHIBIT 7

Average percent change from baseline
TRT = B

| VISIT | N Obs | N | Mean |
|---|---|---|---|
| 1 | 7 | 7 | 1.7857143 |
| 2 | 7 | 7 | 0 |
| 3 | 7 | 7 | 11.9047619 |
| 4 | 7 | 7 | 15.2777778 |
| 5 | 7 | 7 | 26.1904762 |
| 6 | 7 | 7 | 27.7777778 |
| 7 | 7 | 7 | 29.7619048 |
| 8 | 7 | 7 | 47.6190476 |
| 9 | 7 | 7 | 42.4603175 |
| 10 | 7 | 7 | 38.0952381 |
| 11 | 7 | 7 | 38.0952381 |
| 12 | 7 | 7 | 54.3650794 |
| 13 | 7 | 7 | 55.9523810 |
| 14 | 7 | 7 | 55.9523810 |
| 15 | 7 | 7 | 59.1269841 |
| 16 | 7 | 7 | 55.9523810 |
| 17 | 7 | 7 | 57.5396825 |
| 18 | 7 | 7 | 72.0238095 |
| 19 | 7 | 7 | 70.2380952 |
| 20 | 7 | 7 | 54.3650794 |
| 21 | 7 | 7 | 62.6984127 |
| 22 | 7 | 7 | 64.2857143 |
| 23 | 7 | 7 | 64.2857143 |
| 24 | 7 | 7 | 80.1587302 |
| 25 | 7 | 7 | 65.8730159 |
| 26 | 7 | 7 | 60.7142857 |
| 27 | 7 | 7 | 76.7857143 |
| 28 | 7 | 7 | 73.2142857 |
| 29 | 7 | 4 | 125.0000000 |

A statistical analysis of the total data for all subjects for all of the 29 days of active treatment (A) versus control treatment (B) using an ANOVA model shows that there is a highly significant overall effect due to the active treatment (A) vs. control (p<0.0001), with and an increased overall sebosupressive effect over time (p<0.02; Day 1 to Day 29), with no interaction effect between treatment and time.

Moreovr, a questionnaire administered to the subjects at the end of the study indicated substantial oil control activity for the product containing the antihistamine but not for the control.

For example, at the conclusion of the study, 7 of 8 subjects treated with the antihistamine lotion rated their skin as less oily from daily use of the lotion while only 3 of 7 subjects that used the control rated their skin as less oily. The results of this study and the previous three studies summarized above clearly demonstrate that histamine antagonists and specifically, H-1 receptor antagonists have the ability to reduce, suppress, control and or prevent the production of sebum when topically applied to subjects suffering from oily facial skin and oily skin associated with acne.

In order to determine if the sebosupressive activity of topically applied H-1 antagonists have synergistic or additive activity when combined with acne treatments, a topical formulation containing both diphenhydramine HCl, 2% and salicylic acid, 2% was evaluated on acne patients with moderately oily facial skin. The formulas for this acne and oil control treatment gel as well as the positive control formula containing the same acne active and base, but no H-1 antagonist, are shown in Exhibit 8 and Exhibit 9 below.

### EXHIBIT 8

### Acne/Oil Control Lotion

### Formula #26-79 A

| Ingredient | % w/w |
|---|---|
| Diphenhydramine Hydrochloride | 2.00 |
| Salicylic Acid | 2.00 |
| Natrosol HHR 250, (1% Solution) | 45.00 |
| Ethoxydiglycol (Trivalin SF) | 6.00 |
| 1,3-Butylene Glycol | 6.00 |
| Glycerin | 2.00 |
| Ethanol SD 40, 190 proof | 20.00 |
| Glucam E-20 (Amerchol) | 2.00 |
| Phenonip | 0.60 |
| Citrus Sea fragrance 40080T | 0.07 |
| Disodium EDTA | 0.05 |
| Deionized Water | 14.28 |
| Total | 100.00 |

### EXHIBIT 9

### Acne/Oil Control Lotion

### Formula #26-79 B

(Positive Control))

| Ingredient | % w/w |
|---|---|
| Salicylic Acid | 2.00 |
| Natrosol HHR 250, (1% Solution) | 45.00 |
| Ethoxydiglycol (Trivalin SF) | 6.00 |
| 1,3-Butylene Glycol | 6.00 |
| Glycerin | 2.00 |
| Ethanol SD 40, 190 proof | 20.00 |
| Glucam E-20 (Amerchol) | 2.00 |
| Phenonip | 0.60 |
| Citrus Sea fragrance 40080T | 0.07 |
| Disodium EDTA | 0.05 |
| Deionized Water | 16.28 |
| Total | 100.00 |

The fifth pilot clinical study evaluated twice daily (morning and evening) topical facial application of a clear lotion/gel formulation containing diphenhydramine HCl, 2% and salicylic acid, 2% (Formula A; Exhibit 8) versus a positive control lotion/gel with no antihistamine and 2% salicylic acid (Formula B; Exhibit 9). A randomized assignment of formulation A or B was made in this double-blind, positive control pilot study. Subjects were screened for eligibility based upon history of oily skin associated with mild-moderate acne, agreement to use no other topical products during the study other than the test products and a mild facial cleansing bar provided, as well as answers to a questionnaire of various inclusion and exclusion criteria. A total of 18 subjects (9-test product; 9-control product) treated their facial skin twice daily for 8 weeks. Subjects were graded for acne (papules, nodules, pustules, closed and open comedones, etc.) on the cheek, chin and forehead area by a trained clinician and facial oiliness was measured by the test subjects utilizing the self-assessment criteria described above for the other clinical studies as well as by the clinician.

At the end of 4 weeks, subjects treated with either the combined active treatment (Formula A) or the positive control treatment (Formula B) showed approximately 40% reduction in acne papules (p<0.01). However, only Formula A, containing both the histamine antagonist and salicylic acid, showed a statistically significant and meaningful reduction in facial oiliness (25.6% average reduction; p<0.004) based on the clinician assessment (4 weeks compared to baseline). Additionally, the patient self-assessment diary data showed increased sebosuppressive activity over time with average reductions of 8.3%, 41.7% & 44.4% for weeks two, three, and four of the study (p=0.22) while the control formulation showed reductions of 2.8%, 13.2%, & 8.9% for these same time intervals.

The series of five clinical studies outlined above show a consistent pattern of sebosuppressive activity for the topical application of histamine antagonists when applied to excessively oily skin, whether associated with disease (e.g., acne) or cosmetically oily facial skin.

Seborrhea or seborrheic dermatitis is another skin disease thought to be associated with abnormal function of the sebaceous glands. It occurs in areas where there are large numbers of sebaceous glands and is characterized by flaking of the skin and red, mildly inflammatory patches. Seborrhea is most common in the hair (a form of dandruff), scalp margins, eyebrows, naso-labial folds, external ear canals, posterior auricular fold, and prestemal area.

Several subjects with bilateral seborrheic dermatitis of the eyebrows, naso-labial folds and posterior auricular folds were treated with Formula A (Exhibit 8) on one side of the face and Formula B (Exhibit 9) on the other side of the face for up to two weeks. Clinical observations of the subjects thus treated indicated superior efficacy for the treatment containing a histamine antagonist (Formula A) based on clinical assessment of the comparative reduction in scaling and erythema.

### Ancillary Actives

The compositions of the present invention can further comprise one or more ancillary active ingredients capable of functioning via a different mode of action in order to enhance the sebosupressive activity of the histamine antagonist(s) and/or to provide other benefits. These ancillary actives may be combined with the histamine antagonist in a suitable dermatological or cosmetic carrier to 1) further reduce the oily or shiny appearance of skin, 2) provide anti-acne benefits to complement the sebosupressive activity of the histamine antagonist, and 3) provide additional benefits beyond the histamine antagonist effect in the treatment of seborrheic dermatitis, or provide additional cosmetic benefits to facial skin, as follows:

A. Oil Control Agents

The ancillary oil control agents may include materials that absorb sebum on the skin surface, materials that reduce sebum spreadability, and/or sebosupressive agents that are complementary to the sebosupressive effect of the histamine antagonist(s). Nonlimiting examples of sebum absorbing materials are clays (e.g., bentonite), talcs, silicas, starches, oil-absorbing polymers (e.g., Microsponge® and Polytrap® polymers produced by Advanced Polymer Systems). Nonlimiting examples of agents that reduce the spreadability of sebum may include hydroxysultaine compositions described in U.S. Pat. No. 4,534,964; in U.S. Pat. No. 4,529,588, and silicone polymers described in U.S. Pat. No.4,515,784.

Nonlimiting examples of ancillary sebosupressive agents are niacinamide, lipase inhibitors, antiandrogens, estrogens or isoflavone estrogen mimetics, 5-alpha reductase inhibitors, certain antifungal compounds, e.g., 1-(1,3-Dioxolan-2-ylmethyl)-1H-imidazoles, zinc salts, salicylate esters, benzoic acid esters, dehydroacetic acid and salts thereof, ethyllinoleate, triethylcitrate, pygeum extract and botanical extracts containing one or more of the aforementioned compounds as described in the article on “Sebosupression”, Cosmetics & Toiletries, vol. 102, pp.140-146 (April 1987).

B. Anti-Acne Agents

Compositions of the present invention can be used to treat acne by reducing, preventing or arresting the acne process and/or clearing or healing existing lesions and can contain ancillary anti-acne active(s) selected from antimicrobial agents, antiandrogens, and comedolytic/keratolytic agents.

Nonlimiting examples of comedolytic and keratolytic agents useful in the present invention are selected from the group of salicylic acid, resorcinol, sulfur, retinoids, lactic acid and glycolic acid. Retinoids includes retinoic acid (all-trans retinoic acid and/or 1 3-cis-retinoic acid), retinal, retinol, and retinyl esters. Nonlimiting examples of antimicrobial agents includes benzoyl peroxide, erythromycin, tetracycline, clindamycin, azelaic acid, adapalene, and tazarotene. Nonlimiting examples of antiandrogens include cyproterone acetate, drospirenone, spironolactone, dienoestrol diacetate and flutamide.

C. Seborrheic Dermatitis Actives

Compositions of the present invention can be used to treat seborrheic dermatitis by reducing or inhibiting the underlying sebaceous activity to help reduce or eliminate the seborrhea plaques which are characterized by scaling and inflammation. Treatment compositions of this invention can contain ancillary active ingredients that assist in reducing the scaling via a keratolytic effect, by reducing inflammation by steroidal or non-steroidal anti-inflammatory activity, by antimicrobial/anti-dandruff activity or by some combination of these effects.

Nonlimiting examples of ancillary actives include coal tar and coal tar extracts, sulfur, resorcinol, salicylic acid, lactic acid, glycolic acid and other keratolytic alpha-hydroxy and beta-hydroxy acids; ketoconazole, selenium sulfide, zinc pyrithione and other antimicrobials active against Pityrosporum sp.; steroidal anti-inflammatory agents, e.g., corticosteroids such as hydrocortisone, clobetasol valerate, dexamethasone, triamcinolone acetonide, betamethasone and non-steroidal anti-inflammatory agents, e.g., piroxicam, fendosal, diclofenac, flufenamic acid, naproxen, ibuprofen, and phenylbutazone.

D. Other Actives

Compositions of the present invention are useful for reducing, inhibiting or eliminating sebum and the negative effect of sebum on the skin. Oily skin, even when not associated with acne or seborrhea, is a particular problem for individuals afflicted with oily facial skin. Accumulation of oil on the face leads to a shiny appearance and the feeling of greasy, unclean skin. This necessitates, frequent washing and attempts throughout the day to keep the facial skin clean.

Compositions of the present invention when applied to facial skin greatly diminish or even eliminate the cosmetic problems associated with oily facial skin.

It is expected that ancillary actives or additives could be added to the composition of the present invention for treatment of other cosmetic facial problems in addition to oily skin. For example, individuals with excessively oily skin or combination skin that includes areas of facial skin that are oily and other areas that are normal or dry, may also have facial photodamage and other signs of skin aging that may include fine lines and wrinkles, age spots and uneven skin pigmentation, rough or uneven skin surface texture, and areas of dryness or blotchiness. Therefore, nonlimiting examples of other cosmetic additives may include skin whiteners or lighteners, e.g., kojic acid, arbutin, ascorbic acid and ascorbate salts; anti-aging ingredients such as alpha-hydroxy acids, beta-hydroxy acids, alpha-keto acids, beta-keto acids, e.g., lactic acid, glycolic acid, and retinoids, e.g., tretinoin, retinal acetate, retinol; antioxidants, e.g., tocopherol, tea polyphenols, alpha bisabolol; and skin barrier repair ingredients, e.g., sphingosine, phytosphingosine and other pro-ceramide and ceramide compounds, linoleic acid or other essential fatty acids, proteins, hydrolyzed proteins, and amino acids.

### Dermatologically & Cosmeticly Acceptable Carriers Or Vehicles

The carrier vehicle for the present invention is designed to deliver the histamine antagonist and any ancillary active into the skin at the appropriate concentration. The carrier can act as a diluent, solvent, dispersant, penetrant or the like for the histamine antagonist and the ancillary active(s) to ensure that the composition can be applied and distributed evenly over the areas of sebaceous activity at an appropriate concentration.

Suitable carriers include conventional or otherwise known carriers that are cosmetically and dermatological acceptable and are physically and chemically compatible with the active components. The carrier vehicle of the present invention includes a wide variety of product forms that are known in the art.

The carrier vehicle may comprise an aqueous, anhydrous, or aqueous alcoholic solution, a water-in-oil emulsion, an oil-in-water emulsion, a microemulsion, an aqueous gel, a hydroalcoholic gel, an anhydrous gel, a serum, a suspension of solid particles or a dispersion of vesicles, microcapsules or microparticles. The carrier vehicle may be a solid, semisolid, liquid, or aerosol composition.

The carrier vehicle may contain one or more ingredients selected from the group consisting of acidifying agents, alkalizing agents, aerosol propellants, antimicrobial agents, antioxidants, buffering agents, chelating agents, coloring agents, defoaming agents, dispersing agents, emollients, emulsifying agents, humectants, fragrances, moisturizers, penetration enhancers, polymers, preservatives, suspending agents, surfactants, thickening agents and water, alcohol, or other solvents.

While the examples set forth above illustrate specific embodiments of the invention and are considered non-limiting examples with variations and modifications thereof all being within the spirit and purview of this invention.

1. A method of reducing sebaceous gland activity in the skin of a human patient susceptible to having excessive or abnormal sebaceous gland activity which comprises the step of administering to said skin a topical composition consisting essentially of:
(a) a safe and effective amount of at least one material selected from the class of histamine antagonists and pharmaceutically-acceptable salts thereof, and
(b) a dermatological-acceptable carrier vehicle.

2. The method of claim 1 wherein the subsequent treatment of one or more of the conditions of acne, seborrhea, and seborrheic dermatitis is enhanced as compared to said treatment lacking such administration.

3. The method of claim 1 wherein the patient has oily or combination of oily to dry skin,

4. The method of claim 3 wherein the skin is associated with large pores, closed or open comedones and or fine lines or wrinkles.

5. A composition which when applied to the skin of a human patient susceptible to having excessive or abnormal sebaceous gland activity reduces said activity, said composition consisting essentially of:
(a) a safe and effective amount of at least one material selected from the class of histamine antagonists and pharmaceutically-acceptable salts thereof; and
(b) a dermatological-acceptable carrier vehicle.

6. The composition of claim 5 wherein the vehicle is also cosmetically acceptable.

7. The composition of claim 5 wherein the vehicle is also pharmaceutically acceptable.

8. The composition of claim 5 wherein the histamine antagonist is a histamine sub.1 receptor inhibitor and capable of inhibiting the contraction of smooth muscle induced by the administration of histamine or inhibiting the release of histamine by stimulated mast cells.

9. The composition of claim 5 wherein the histamine antagonist is a heterocyclic or a nitrogen compound having at least one benzene ring.

10. The composition of claim 5 wherein the total amount of the histamine antagonist compound or compounds ranges from about 0.000001 to 20% by weight relative to the total weight of the composition.

11. The composition of claim 5 wherein the vehicle comprises an aqueous, anhydrous, or aqueous alcoholic solution, a water-in-oil emulsion, an oil-in-water emulsion, a microemulsion, an aqueous gel, a hydroalcoholic gel, an anhydrous gel, a serum, a suspension of solid particles or a dispersion of vesicles, microcapsules or microparticles in a solid, semi-solid, liquid, or aerosol form.

12. The composition of claim 5 containing one or more additional sebosupressive agents selected from the group consisting of a lipase inhibitor, antiandrogens, estrogens or isoflavone estrogen mimetics, 5-alpha reductase inhibitors, certain antifungal compounds, e.g., 1-(1,3-Dioxolan-2-ylmethyl)-1H-imidazoles, zinc salts, salicylate esters, benzoic acid esters, dehydroacetic acid, ethyllinoleate, triethylcitrate, niacinamide, pygeum extract and botanical extracts containing one or more of the aforementioned compounds.

13. The composition of claim 5 containing at least one additional pharmaceutically, dermatological, or cosmeceutically active ingredients selected from the group consisting of skin lighteners and whiteners or skin depigmenting ingredients, anti-aging ingredients including alpha-hydroxy acids, beta-hydroxy acids, alpha-keto acids, beta-keto acids or retinoids, antioxidants, and skin barrier repair compounds including phytosphingosine and ceramide compounds, essential fatty acids, proteins, hydrolyzed proteins and amino acids.

14. The composition of claim 5 containing one or more additional pharmaceutically or dermatological active ingredient for the treatment or prevention of acne selected from the group consisting of salicylic acid, lactic acid, glycolic acid, colloidal sulfur, resorcinol, retinoic acid (tretinoin or isotretinoin), retinol, retinaldehyde, retinyl esters, selenium disulphide, benzoyl peroxide, azelaic acid, clindamycin, erythromycin, adapalene, and tazarotene.

15. The composition of claim 5 containing at lease one additional pharmaceutically or dermatological active ingredient for the treatment or prevention of seborrhea or seborrheic dermatitis selected from the group consisting of sulfur, resorcinol, an alpha-hydroxy acid, a beta-hydroxy acid, ketoconazole, a steroidal or non-steroidal anti-inflammatory, an antibiotic, coal tar or coal tar extract, urea, selenium sulfide, and/or zinc pyrithione.

16. The composition of claim 5 wherein the carrier base or vehicle contains at least one ingredient selected from the group consisting of acidifying agents, alkalizing agents, aerosol propellants, antimicrobial agents, antioxidants, buffering agents, chelating agents, coloring agents, defoaming agents, dispersing agents, emollients, emulsifying agents, humectants, fragrances, moisturizers, penetration enhancers, polymers, preservatives, suspending agents, surfactants, thickening agents and water, alcohol, or other solvents.
